(19) United States
(12) Patent Application Publication
(10) Pub. No.: US 2026/0038117 A1
(43) Pub. Date: Feb. 5, 2026

(54) MEDICAL IMAGE PROCESSING METHOD AND APPARATUS, ELECTRONIC DEVICE, STORAGE MEDIUM, AND PROGRAM PRODUCT

(71) Applicant: GE Precision Healthcare LLC, Waukesha, WI (US)

(72) Inventor: Yizhen Zhang, Wuxi (CN)

(21) Appl. No.: 19/288,972

(22) Filed: Aug. 1, 2025

(30) Foreign Application Priority Data
Aug. 2, 2024 (CN) 202411055951.9

(51) Int. Cl. G06T 7/00; A61B 8/00; G06T 1/00; G06V 10/74; G16H 30/20

(52) U.S. Cl. CPC G06T 7/0012; G06T 1/0028; G06V 10/761; G16H 30/20; A61B 8/5292; G06T 2201/0065; G06T 2201/0202; G06T 2207/30168; G06V 2201/03

(57) ABSTRACT

A medical image processing method and apparatus, an electronic device, a storage medium, and a program product. The method includes extracting a current digital watermark in a target medical image; acquiring an original digital watermark corresponding to the target medical image; and comparing the current digital watermark with the original digital watermark, and determining quality information of the target medical image.

### CROSS-REFERENCE TO RELATED APPLICATION

This application claim priority to Chinese Patent Application No. 202411055951.9, which was file on Aug. 2, 2024 at the Chinese Patent Office. The entire contents of the above-listed application are incorporated by reference herein in their entirety.

### TECHNICAL FIELD

The present disclosure relates to the field of image technology, and in particular, to a medical image processing method and apparatus, an electronic device, a computer-readable storage medium, and a computer program product.

### BACKGROUND

The image quality of medical images is a cornerstone for ensuring accurate diagnosis. With the rapid development of medical informatization, the approaches of acquiring, storing and transmitting medical image data are also continuously evolving. Although conventional medical image formats, such as a DICOM (Digital Imaging and Communications in Medicine) format, provide detailed image information and excellent image quality in medical diagnosis, they exhibit large file sizes and require specific software or hardware support for reading and parsing, which may not be ideal in resource-constrained emergency scenarios. For example, in situations such as emergency care, online hospitals, and home diagnostics, doctors may not have the means to view the medical images in DICOM format.

To address this issue, medical images are often converted to a JPEG (Joint Photographic Experts Group) format for storage and transmission. The JPEG format, with its smaller file size and broad compatibility, allows medical images to be easily read across various devices and platforms, which greatly facilitates the rapid sharing and access of medical images. However, the JPEG format employs lossy compression technology. Although this compression approach reduces the file size, it may also cause damage to the image quality, particularly after multiple compressions, it may affect the diagnostic values of medical images. In other words, it is uncertain to the doctors whether the medical images retain sufficient diagnostic values after transmission and compression.

### SUMMARY OF THE INVENTION

The present disclosure provides a technical solution for processing medical images.

According to an aspect of the present disclosure, provided is a medical image processing method, comprising:
extracting a current digital watermark in a target medical image;
acquiring an original digital watermark corresponding to the target medical image; and
comparing the current digital watermark with the original digital watermark, and determining quality information of the target medical image.

In a possible implementation, the extracting a current digital watermark in a target medical image comprises:
in response to an instruction to open the target medical image, automatically extracting the current digital watermark in the target medical image.

In a possible implementation, the determining quality information of the target medical image comprises:
determining a similarity between the current digital watermark and the original digital watermark; and
determining the quality information of the target medical image based on the similarity.

In a possible implementation, the quality information comprises validity information of the target medical image;
and the determining the quality information of the target medical image based on the similarity comprises:
in response to the similarity being greater than or equal to a first preset similarity threshold, determining that the validity information of the target medical image is valid; or,
in response to the similarity being less than the first preset similarity threshold, determining that the validity information of the target medical image is invalid.

In a possible implementation, the method further comprises:
determining whether an image quality value of the target medical image is less than a quality threshold; and
when the image quality value is less than the quality threshold, the quality information comprising a low quality cause of the target medical image.

In a possible implementation, when the image quality value is less than the quality threshold, the determining quality information of the target medical image comprises:
in response to the similarity being greater than or equal to a second preset similarity threshold, determining that the low quality cause of the target medical image comprises an imaging cause;
or,
in response to the similarity being less than the second preset similarity threshold, determining that the low quality cause of the target medical image comprises a transmission cause and/or a compression cause.

In a possible implementation, the current digital watermark is embedded in a region of interest of the target medical image, and the size of the current digital watermark matches the size of the region of interest.

In a possible implementation, at least two regions of interest are comprised; and
the current digital watermark is embedded in a merged region of the at least two regions of interest,
or,
the current digital watermark is embedded in the at least two regions of interest, respectively.

In a possible implementation, the quality information comprises:
region quality information in one-to-one correspondence with the at least two regions of interest.

In a possible implementation, the shape of the original digital watermark corresponds to the shape of the target medical image.

In a possible implementation, the method further comprises:
simultaneously displaying the target medical image and the quality information.

In a possible implementation, the target medical image is obtained through transmission.

According to an aspect of the present disclosure, provided is a medical image processing method, comprising:
acquiring a target medical image;
embedding an original digital watermark in the target medical image; and
transmitting the target medical image.

In a possible implementation, the acquiring a target medical image comprises:
performing imaging on a subject to be imaged to obtain imaging data; and
processing the data to generate the target medical image.

In a possible implementation, the embedding an original digital watermark in the target medical image comprises:
determining a region of interest of the target medical image, and embedding the original digital watermark in the region of interest in such a manner that the size of the original digital watermark matches the size of the region of interest.

According to one aspect of the present disclosure, provided is a medical image processing apparatus, comprising:
an extraction module, used to extract a current digital watermark in a target medical image;
a first acquisition module, used to acquire an original digital watermark corresponding to the target medical image; and
a first determination module, used to compare the current digital watermark with the original digital watermark to determine quality information of the target medical image.

In a possible implementation, the extraction module is used to:
in response to an instruction to open the target medical image, automatically extract the current digital watermark in the target medical image.

In a possible implementation, the first determination module is used to:
determine a similarity between the current digital watermark and the original digital watermark; and
determine the quality information of the target medical image based on the similarity.

In a possible implementation, the quality information comprises validity information of the target medical image;
the first determination module is used to:
in response to the similarity being greater than or equal to a first preset similarity threshold, determine that the validity information of the target medical image is valid; or,
in response to the similarity being less than the first preset similarity threshold, determine that the validity information of the target medical image is invalid.

In a possible implementation, the apparatus further includes:
a second determination module, used to determine whether an image quality value of the target medical image is less than a quality threshold; and
when the image quality value is less than the quality threshold, the quality information comprises a low quality cause of the target medical image.

In a possible implementation, when the image quality value is less than the quality threshold, the first determination module is used to:
in response to the similarity being greater than or equal to a second preset similarity threshold, determine that the low quality cause of the target medical image comprises an imaging cause;
or,
in response to the similarity being less than the second preset similarity threshold, determine that the low quality cause of the target medical image comprises a transmission cause and/or a compression cause.

In a possible implementation, the current digital watermark is embedded in a region of interest of the target medical image, and the size of the current digital watermark matches the size of the region of interest.

In a possible implementation, at least two regions of interest are comprised; and
the current digital watermark is embedded in a merged region of the at least two regions of interest,
or,
the current digital watermark is embedded in the at least two regions of interest, respectively.

In a possible implementation, the quality information comprises:
region quality information in one-to-one correspondence with the at least two regions of interest.

In a possible implementation, the shape of the original digital watermark corresponds to the shape of the target medical image.

In a possible implementation, the apparatus further includes:
a display module, used to simultaneously display the target medical image and the quality information.

In a possible implementation, the target medical image is obtained through transmission.

According to one aspect of the present disclosure, provided is a medical image processing apparatus, comprising:
a second acquisition module, used to acquire a target medical image;
an embedding module, used to embed an original digital watermark in the target medical image; and
a transmission module, used to transmit the target medical image.

In a possible implementation, the second acquisition module is used to:
perform imaging on a subject to be imaged to obtain imaging data; and
process the data to generate the target medical image.

In a possible implementation, the embedding module is used to:
determine a region of interest of the target medical image, and embed the original digital watermark in the region of interest in such a manner that the size of the original digital watermark matches the size of the region of interest.

According to an aspect of the present disclosure, provided is an electronic device, comprising: one or more processors; and a memory for storing executable instructions, wherein the one or more processors are configured to invoke the executable instructions stored by the memory to perform the above method.

According to an aspect of the present disclosure, provided is a computer-readable storage medium, having computer program instructions stored thereon, wherein the computer program instructions, when executed by a processor, implement the above method.

According to an aspect of the present disclosure, provided is a computer program product, comprising computer-readable code or a non-volatile computer-readable storage medium carrying computer-readable code, wherein when the computer-readable code is run in an electronic device, a processor in the electronic device performs the above method.

In the embodiments of the present disclosure, by extracting a current digital watermark in a target medical image, acquiring an original digital watermark corresponding to the target medical image, and comparing the current digital watermark with the original digital watermark to determine quality information of the target medical image, the quality information of the medical image can be provided to a doctor, thereby helping the doctor to determine the validity of the medical image, that is, helping the doctor to determine whether the medical image has a sufficient diagnostic value, which is beneficial to improving the confidence of diagnosis based on the medical image and the accuracy of treatment.

It should be understood that the foregoing general description and the following detailed description are merely exemplary and explanatory and do not limit the present disclosure.

Other features and aspects of the present disclosure will become apparent from the following detailed description of exemplary embodiments provided with reference to the accompanying drawings.

### BRIEF DESCRIPTION OF THE DRAWINGS

The accompanying drawings here are incorporated into the specification, constitute a part of the specification, illustrate embodiments compliant with the present disclosure, and are used together with the specification to describe the technical solutions of the present disclosure.

FIG. 1 illustrates a flowchart of a medical image processing method provided in an embodiment of the present disclosure.

FIG. 2 illustrates a schematic diagram of an original digital watermark corresponding to a rectangular medical image acquired via a linear array probe in the medical image processing method provided in an embodiment of the present disclosure.

FIG. 3 illustrates a schematic diagram of an original digital watermark corresponding to a convex medical image acquired via a convex array probe in the medical image processing method provided in an embodiment of the present disclosure.

FIG. 4 illustrates a schematic diagram of an original digital watermark corresponding to a sector-shaped medical image acquired via a phased array probe in the medical image processing method provided in an embodiment of the present disclosure.

FIG. 5 illustrates a schematic diagram of an original digital watermark corresponding to a region of interest in a medical image acquired via the phased array probe and the convex array probe in the medical image processing method provided in an embodiment of the present disclosure.

FIG. 6 illustrates a schematic diagram of an original digital watermark corresponding to a region of interest in a medical image acquired via the linear array probe in a steered mode in the medical image processing method provided in an embodiment of the present disclosure.

FIG. 7 illustrates a schematic diagram of an original digital watermark corresponding to a region of interest in a medical image acquired via the linear array probe in a non-steered mode in the medical image processing method provided in an embodiment of the present disclosure.

FIG. 8 illustrates a schematic diagram of an original medical image in the medical image processing method provided in an embodiment of the present disclosure.

FIG. 9 illustrates a schematic diagram of a region of interest in the original medical image.

FIG. 10 illustrates a schematic diagram of an original digital watermark corresponding to the region of interest in the original medical image.

FIG. 11 illustrates a schematic diagram of patterns of an original digital watermark in the medical image processing method provided in an embodiment of the present disclosure.

FIG. 12 illustrates a schematic diagram of an original medical image and a region of interest thereof in the medical image processing method provided in an embodiment of the present disclosure.

FIG. 13 illustrates a schematic diagram of a medical image after transmission and compression and a region of interest thereof in the medical image processing method provided in an embodiment of the present disclosure.

FIG. 14 illustrates another schematic diagram of the medical image after transmission and compression and the region of interest thereof in the medical image processing method provided in an embodiment of the present disclosure.

FIG. 15 illustrates another flowchart of the medical image processing method provided in an embodiment of the present disclosure.

FIG. 16 illustrates a schematic diagram of a medical image displayed on a terminal device corresponding to a medical image examiner in the medical image processing method provided in an embodiment of the present disclosure.

FIG. 17 illustrates a schematic diagram of a screenshot-protected image in the medical image processing method provided in an embodiment of the present disclosure.

FIG. 18 illustrates a block diagram of a medical image processing apparatus provided in an embodiment of the present disclosure.

FIG. 19 illustrates another block diagram of the medical image processing apparatus provided in an embodiment of the present disclosure.

FIG. 20 illustrates a block diagram of an electronic device 1900 provided in an embodiment of the present disclosure.

### DETAILED DESCRIPTION

Various exemplary embodiments, features, and aspects of the present disclosure will be described in detail below with reference to the accompanying drawings. The same reference numerals in the drawings represent elements having the same or similar functions. While various aspects of the embodiments are illustrated in the accompanying drawing, the accompanying drawings are not necessarily drawn to scale unless specifically stated.

The word “exemplary” dedicated herein means “used as an example or an embodiment, or is illustrative”. Any of the embodiments illustrated herein as “exemplary” is not necessarily interpreted as being superior to or better than other embodiments.

The term “and/or” herein is merely to describe the associations of associated objects, indicating that there may be three kinds of relationships. For example, A and/or B may indicate three situations, i.e., A exists alone, A and B exist simultaneously, or B exists alone. In addition, the term “at least one” herein indicates any one of a plurality or any combination of at least two of a plurality. For example, at least one of A, B, and C may indicate any one or more elements selected from the group consisting of A, B, and C.

In addition, in order to better illustrate the present disclosure, numerous specific details are given in the detailed description below. It should be understood by those skilled in the art that the present disclosure can be implemented without some of the specific details. In some examples, the methods, means, elements, and circuits well known to those skilled in the art are not described in detail in order to highlight the main idea of the present disclosure.

The embodiments of the present disclosure provide a medical image processing method. By extracting a current digital watermark in a target medical image, acquiring an original digital watermark corresponding to the target medical image, and comparing the current digital watermark with the original digital watermark to determine quality information of the target medical image, the quality information of the medical image can be provided to a doctor, thereby helping the doctor to determine the validity of the medical image, that is, helping the doctor to determine whether the medical image has a sufficient diagnostic value, which is beneficial to improving the confidence of diagnosis based on the medical image and the accuracy of treatment.

A medical image processing method provided in an embodiment of the present disclosure will be described in detail below with reference to the accompanying drawings.

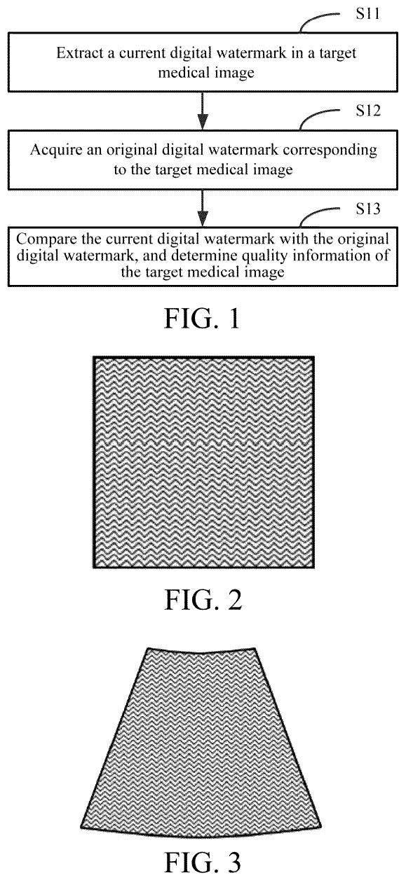

FIG. 1 illustrates a flowchart of a medical image processing method provided in an embodiment of the present disclosure. In a possible implementation, an executing entity of the medical image processing method may be a medical image processing apparatus. For example, the medical image processing method may be executed by a terminal device or other electronic devices. The terminal device may be a mobile phone, a tablet computer, a personal computer (PC), a notebook computer, or the like. In a possible implementation, the executing entity of the medical image processing method may be a terminal device corresponding to an observer. For example, the observer may be a doctor, a patient, or other image reader. The terminal device corresponding to the observer may be any terminal device that can be operated by the observer. In some possible implementations, the medical image processing method may be implemented by a processor by invoking computer-readable instructions stored in a memory. As shown in FIG. 1, the medical image processing method includes step S11 to step S13.

In step S11, a current digital watermark in a target medical image is extracted.

In step S12, an original digital watermark corresponding to the target medical image is acquired.

In step S13, the current digital watermark is compared with the original digital watermark to determine quality information of the target medical image.

In the embodiment of the present disclosure, the target medical image may be any medical image that a user (e.g., a doctor, a patient, or other image readers) wishes to view. The medical image may represent an image of an internal structure of a patient's body acquired by a medical image device (e.g., an ultrasonic scanning device, an X-ray device, a magnetic resonance imaging (MRI) device, or the like).

In a possible implementation, the image type of the target medical image may be an image. In this implementation, the target medical image may be a static medical image, for example, may be a snapshot of an internal structure of a human body captured at a specific moment. In this implementation, the target medical image may be two-dimensional, or may be a three-dimensional structure reconstructed from a plurality of parallel two-dimensional images.

In another possible implementation, the image type of the target medical image may be a video. Unlike static images, in this implementation, the target medical image may be a video composed of a series of consecutive images, showing the change of the internal structure of the human body over time. In this implementation, the target medical image in the form of a video may provide dynamic information such as hemodynamics, movements of organs.

In a possible implementation, the target medical image may be a medical image acquired by a handheld medical image device. The handheld medical image device may be a portable, hand-operable device that enables medical professionals to perform rapid diagnostics in various environments (including clinical settings, field locations, or patient bedsides). For example, the handheld medical image device may be a handheld ultrasound scanner (also referred to as a portable ultrasound device), a handheld X-ray device, a handheld magnetic resonance imaging device, or the like.

In another possible implementation, the target medical image may be a medical image acquired by a non-handheld medical image device. The non-handheld medical image devices are typically stationary and may be designed for use in hospitals, clinics, or specialized image centers. For example, the non-handheld medical image device may be an ultrasound device, an X-ray machine, a computed tomography (CT) device, a magnetic resonance imaging device, a positron emission tomography (PET) device, or the like.

In the embodiments of the present disclosure, a user (e.g., a doctor) may view the target medical image in various ways.

In a possible implementation, the user may view the target medical image through a mobile application. The mobile application may be an application specifically designed for mobile devices (e.g., smartphones and tablet computers), which may allow users to access medical images anytime and anywhere. The mobile application may have a touch interface which supports multi-touch operations to facilitate the user to zoom, pan and view image details.

In another possible implementation, the user may view the target medical image through a .exe file on a PC. The .exe file is an executable program file that can run on the Windows operating system. The user may install such software on a personal computer or workstation for viewing, processing and analyzing medical images.

In another possible implementation, the user may view the target medical image through a website. In this implementation, through an online platform or service accessed via the Internet, the user may view the medical image in a web browser. This approach allows the user to access images on different devices, provided that these devices can connect to the Internet and run compatible browsers.

In another possible implementation, the user may view the target medical image through an image viewer plug-in (e.g., JPEG viewer plug-in). As an extension of existing software (e.g., an image viewer or browser), the image viewer plug-in may enhance the functionality of the software, enabling the software to display and process medical image formats, such as DICOM or JPEG.

Digital watermarking technology achieves copyright protection, authentication, and information embedding of contents without affecting the perceptual quality of original signals, by embedding imperceptible hidden information or unique identifiers into host signals such as images, audios, or videos. This technology is an effective means for creators to confirm the ownership of works, monitor the distribution of works, and ensure that the integrity of digital contents is maintained.

In the embodiments of the present disclosure, the current digital watermark in the target medical image is extracted. The current digital watermark may be extracted from the target medical image by using reverse engineering of a digital watermark embedding algorithm. In the case where the target medical image is a video, the current digital watermark may be extracted from the target medical image frame by frame.

In the embodiments of the present disclosure, the original digital watermark may represent a digital watermark embedded in the target medical image in a digital watermark embedding process of the target medical image. The current digital watermark may represent a digital watermark extracted from the target medical image. For example, the current digital watermark may be a digital watermark extracted from the target medical image again after the target medical image has undergone processes such as transmission, storage, and compression.

In a possible implementation, the extracting a current digital watermark in a target medical image includes: in response to an instruction to open the target medical image, automatically extracting the current digital watermark in the target medical image.

In this implementation, when a user (e.g., a doctor) opens the target medical image, an extraction process of the current digital watermark in the target medical image and a subsequent quality detection process are automatically triggered. That is, for the user, only by opening the target medical image, the quality detection process of the target medical image can be triggered and the quality information of the target medical image can be obtained, without the user opening another software to perform quality detection on the target medical image. This implementation provides immediate quality feedback to the user, so that the doctor can quickly know whether the target medical image is suitable for clinical diagnosis.

In a possible implementation, the shape of the original digital watermark corresponds to the shape of the target medical image.

In this implementation, when the original digital watermark is embedded, the shape of the original digital watermark may match the overall shape of the target medical image. For example, if the target medical image is rectangular, the original digital watermark may also be rectangular. If the target medical image has specific edges or feature shapes, then the design of the original digital watermark may also adapt to these features accordingly.

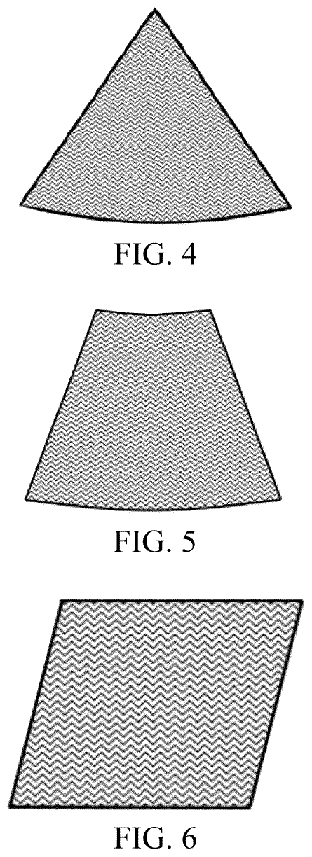

For example, in B-mode scanning, common types of probes include linear array probes, convex array probes, and phased array probes. The linear array probe may generate a rectangular imaging frame, which may be used for examinations of superficial organs, blood vessels, abdomen, etc. FIG. 2 illustrates a schematic diagram of an original digital watermark corresponding to a rectangular medical image acquired via a linear array probe in the medical image processing method provided in an embodiment of the present disclosure. The convex array probe may generate a convex imaging frame, which may be suitable for larger imaging areas, such as abdominal, gynecological and cardiac examinations. FIG. 3 illustrates a schematic diagram of an original digital watermark corresponding to a convex medical image acquired via a convex array probe in the medical image processing method provided in an embodiment of the present disclosure. The phased array probe may generate a sector-shaped imaging frame, which may be used for examinations of the heart, neck blood vessels, and superficial organs. FIG. 4 illustrates a schematic diagram of an original digital watermark corresponding to a sector-shaped medical image obtained via a phased array probe in the medical image processing method provided in an embodiment of the present disclosure.

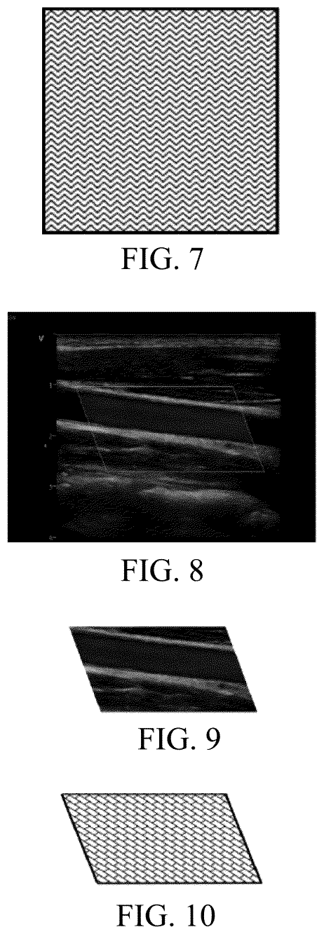

For another example, in an ultrasound image of a blood flow mode, a specific region of interest (ROI) may be set, that is, a specific region is selected in the image for blood flow analysis. The region of interest may be in the shape of a rectangle, a parallelogram, a sector, or the like. The phased array probe and the linear array probe may be used for blood flow detection of the heart and blood vessels, as they may provide good blood flow imaging. The convex array probe may also be used in blood flow modes, especially in abdominal and gynecological applications. FIG. 5 illustrates a schematic diagram of an original digital watermark corresponding to a region of interest in a medical image acquired via the phased array probe and the convex array probe in the medical image processing method provided in an embodiment of the present disclosure. FIG. 6 illustrates a schematic diagram of an original digital watermark corresponding to a region of interest in a medical image acquired via a linear array probe in a steered mode in the medical image processing method provided in an embodiment of the present disclosure. FIG. 7 illustrates a schematic diagram of an original digital watermark corresponding to a region of interest in a medical image acquired via the linear array probe in a non-steered mode in the medical image processing method provided in an embodiment of the present disclosure.

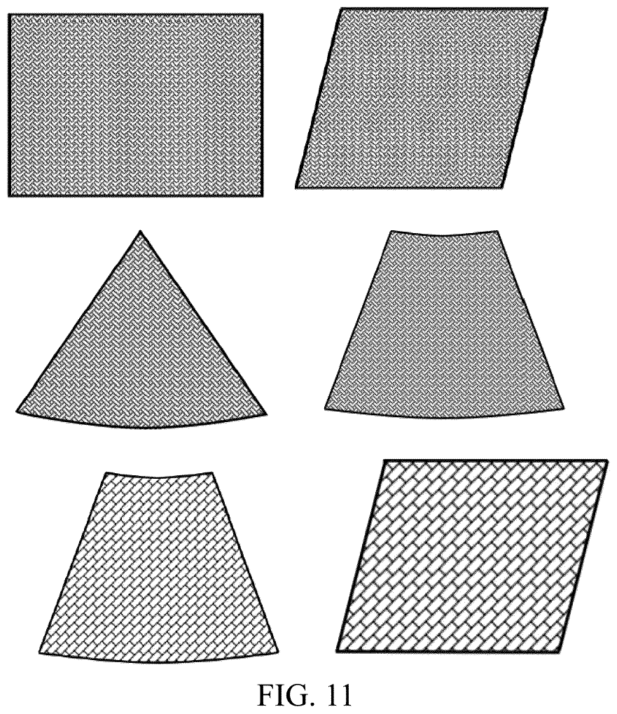

FIG. 8 illustrates a schematic diagram of an original medical image in the medical image processing method provided in an embodiment of the present disclosure. FIG. 9 illustrates a schematic diagram of a region of interest in the original medical image. FIG. 10 illustrates a schematic diagram of an original digital watermark corresponding to the region of interest in the original medical image.

In this implementation, by making the shape of the original digital watermark correspond to the shape of the target medical image, the concealment of the digital watermark can be improved, and the influence on the visual quality of the target medical image can be reduced, so that the doctor is not interfered by the digital watermark during diagnosis. In addition, when it is necessary to extract the digital watermark to verify the integrity or authenticity of the target medical image, the shape-corresponding design can make the extraction process simpler and more accurate.

FIG. 11 illustrates a schematic diagram of patterns of an original digital watermark in the medical image processing method provided in an embodiment of the present disclosure. As shown in FIG. 11, the pattern of the original digital watermark may be a curve texture, a noise texture, a dot matrix texture, a natural texture, or the like. The curve texture may use curve-shaped patterns, such as spiral lines or wavy lines. The curve texture can create complex and imperceptible modes in medical images. The noise texture may represent a texture that simulates natural noise which may be Gaussian noise, salt-and-pepper noise, etc. The dot matrix texture may be a pattern composed of dots which may be a regular dot matrix arrangement or randomly distributed dots. The natural texture may imitate a texture in the natural world, such as wood grain, marble texture.

In the embodiments of the present disclosure, when quality detection needs to be performed on the target medical image, the original digital watermark corresponding to the target medical image may be obtained. In a possible implementation, the original digital watermark corresponding to the target medical image may be retrieved from a storage medium. For example, a database may be queried to find a corresponding original digital watermark based on a unique identifier (e.g., an image ID or a patient ID) of the target medical image. For another example, the shape of the target medical image may be compared with the shape of the digital watermark in the database, and the digital watermark whose shape is consistent with the shape of the target medical image may be determined as the original digital watermark corresponding to the target medical image.

In a possible implementation, the current digital watermark may be compared with the original digital watermark by using a terminal device corresponding to the observer to determine the quality information of the target medical image.

In another possible implementation, the terminal device corresponding to the observer may upload the current digital watermark to a server side, and the server side compares the current digital watermark with the original digital watermark to determine the quality information of the target medical image.

In a possible implementation, the determining quality information of the target medical image includes: determining a similarity between the current digital watermark and the original digital watermark; and determining the quality information of the target medical image based on the similarity.

In this implementation, the quality information of the target medical image may be determined by calculating the similarity between the current digital watermark and the original digital watermark. The similarity measurement methods may include correlation, mean squared error (MSE), peak signal-to-noise ratio (PSNR), etc.

In this implementation, the quality information of the target medical image is determined according to the similarity between the current digital watermark and the original digital watermark, so that the image quality of the target medical image can be accurately evaluated, thereby reducing the risk of misdiagnosis caused by image quality issues.

In another possible implementation, feature matching may be performed on the current digital watermark and the original digital watermark to determine the quality information of the target medical image. In this implementation, the current digital watermark and the original digital watermark may be compared by identifying and comparing feature points or feature vectors in the current digital watermark and the original digital watermark.

In another possible implementation, error rate analysis may be performed on the current digital watermark based on the original digital watermark, thereby determining the quality information of the target medical image. In this implementation, the number of differing bits between the current digital watermark and the original digital watermark, that is, an error rate, may be calculated, thereby determining the quality information of the target medical image.

In another possible implementation, the distribution characteristics of the current digital watermark and the original digital watermark may be compared using a statistical method, such as a histogram, a probability density function, etc., thereby determining the quality information of the target medical image.

In another possible implementation, the features of the current digital watermark and the original digital watermark may be identified and compared using a trained machine learning model to determine the quality information amount of the target medical image.

In a possible implementation, the quality information includes validity information of the target medical image; and the determining the quality information of the target medical image based on the similarity includes: determining that the validity information of the target medical image is valid in response to the similarity being greater than or equal to a first preset similarity threshold, or determining that the validity information of the target medical image is invalid in response to the similarity being less than the first preset similarity threshold.

The validity information of the target medical image being valid may represent that the image quality of the target medical image is good, the target medical image is not tampered with or the quality loss is within an acceptable range. The validity information of the target medical image being invalid may represent that the target medical image is tampered with or severely damaged.

In this implementation, by determining the validity information of the target medical image, the doctor may use only the medical image whose validity information is valid for diagnosis, thereby reducing the possibility of misdiagnosis.

In a possible implementation, the method further includes: determining whether an image quality value of the target medical image is less than a quality threshold; and when the image quality value is less than the quality threshold, the quality information including a low quality cause of the target medical image.

In this implementation, the quality information of the target medical image may further include an image quality value of the target medical image. The resolution, contrast, sharpness, etc. of the target medical image may be quantitatively analyzed to determine the image quality value of the target medical image.

In this implementation, if the image quality value of the target medical image is less than the quality threshold, then the low quality cause of the target medical image may be determined.

For users (e.g., doctors), it is usually difficult to distinguish whether the low quality of the medical image is caused by intrinsic factors such as reverberation and aberration or caused by external factors such as transmission or compression. In this implementation, by identifying the low quality cause of the target medical image, the doctor can more accurately evaluate the diagnostic value of the target medical image, thereby avoiding misdiagnosis caused by image quality issues.

In a possible implementation, when the image quality value is less than the quality threshold, the determining quality information of the target medical image includes: in response to the similarity being greater than or equal to a second preset similarity threshold, determining that the low quality cause of the target medical image includes an imaging cause; or, in response to the similarity being less than the second preset similarity threshold, determining that the low quality cause of the target medical image includes a transmission cause and/or a compression cause.

In this implementation, if the image quality value of the target medical image is less than the quality threshold, and the similarity between the current digital watermark and the original digital watermark is greater than or equal to a second preset similarity threshold, it may be determined that the low quality cause of the target medical image includes the imaging cause. The imaging cause is usually related to technical issues in the process of medical image acquisition, such as reverberation and aberration. In the field of medical imaging, for example, in ultrasonic imaging, the reverberation may be a signal generated after multiple reflections of ultrasonic waves in tissue, which may affect the clarity of images and the accuracy of diagnosis. In optics, the aberration may refer to an aberration of light rays as they pass through an optical system due to imperfections of lenses or other optical components, which may cause imaging distortion. In medical imaging, aberrations may include a spherical aberration, a chromatic aberration, etc., which are unavoidable optical characteristics in an imaging system and may degrade image quality.

If the image quality value of the target medical image is less than the quality threshold, and the similarity between the current digital watermark and the original digital watermark is less than the second preset similarity threshold, it may be determined that the low quality cause of the target medical image includes the transmission cause and/or the compression cause. The transmission reason and the compression reason are usually related to data processing of the medical image during storage and transmission. Compression may result in loss of details of the medical image, while transmission errors may result in data corruption.

In this implementation, a user (e.g., a doctor) is provided with important reference information by determining the low quality cause of the target medical image. By accurately identifying the low quality cause of the target medical image, the doctor can rely more confidently on the medical image for diagnosis, thereby reducing the risk of misdiagnosis.

In a possible implementation, the current digital watermark is embedded in a region of interest of the target medical image, and the size of the current digital watermark matches the size of the region of interest.

In this implementation, the number of the region of interest in the target medical image may be one or at least two.

The region of interest of the medical image is usually a critical region for diagnosis. By embedding the digital watermark only in the regions of interest, the quality assessment of the critical region for diagnosis is not affected even if other parts of the medical image are damaged or have degraded quality. Image quality degradation in non-regions-of-interest may not significantly impact diagnosis; therefore, by embedding the digital watermark only in the regions of interest, unnecessary quality assessment may be reduced (that is, quality assessment of non-regions-of-interests is not required), thereby improving the efficiency of diagnosis processes.

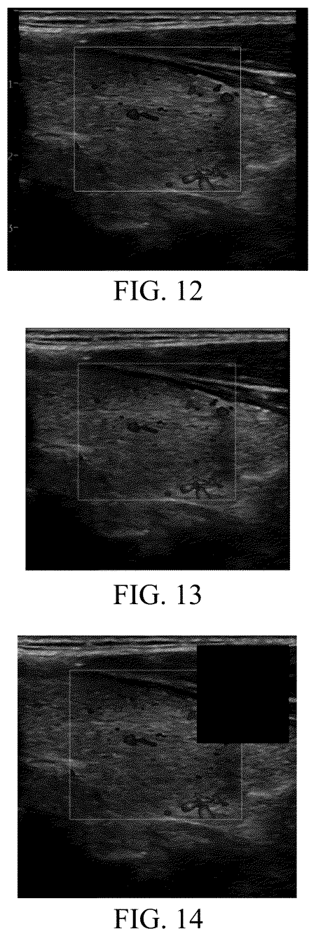

FIG. 12 illustrates a schematic diagram of an original medical image and a region of interest thereof in the medical image processing method provided in an embodiment of the present disclosure. FIG. 13 illustrates a schematic diagram of a medical image after transmission and compression and a region of interest thereof in the medical image processing method provided in an embodiment of the present disclosure. FIG. 14 illustrates another schematic diagram of the medical image after transmission and compression and the region of interest thereof in the medical image processing method provided in an embodiment of the present disclosure. In the example shown in FIG. 14, after the medical image is transmitted and compressed, a part of the image content of the region of interest is missing.

In a possible implementation, at least two regions of interest are included; and the current digital watermark is embedded in a merged region of the at least two regions of interest, alternatively, the current digital watermark is embedded in the at least two regions of interest, respectively.

As an example of this implementation, in the case where the target medical image includes at least two regions of interest, the current digital watermark may be embedded in a merged region of the at least two regions of interest.

In an example, for any two regions of interest in the target medical image, in the case where a distance between the two regions of interest is less than or equal to a preset distance threshold, the current digital watermark may be embedded in a merged region of the two regions of interest.

In this example, when a digital watermark is required to be extracted and verified, the watermark may be extracted in the merged region at one time, which may simplify the current extraction process of the digital watermark and improve the quality detection efficiency of the medical image.

As another example of this implementation, in the case where the target medical image includes at least two regions of interest, the current digital watermark may be embedded in the at least two regions of interest, respectively. The current digital watermark being embedded in the at least two regions of interest, respectively, may represent that each of the at least two regions of interest includes one current digital watermark, that is, the current digital watermarks in different regions of interest are independent of each other.

In an example, for any two regions of interest in the target medical image, in the case where a distance between the two regions of interest is greater than a preset distance threshold, the current digital watermark may be embedded in the two regions of interest respectively.

In this example, each region of interest is embedded with a digital watermark independently, thereby enhancing the protection of different critical regions in the target medical image, so that even if some regions of interest are damaged, watermarks of other regions of interest can still provide integrity verification. The doctor may perform independent analysis on different regions of interest in the target medical image, thereby improving the flexibility of diagnosis.

In a possible implementation, the quality information includes: region quality information in one-to-one correspondence with the at least two regions of interest.

In this implementation, the region quality information may represent quality information corresponding to the region of interest. For each region of interest, region quality information may be provided separately. That is, when the target medical image includes at least two regions of interest, region quality information may be respectively generated for each region of interest. According to the region quality information of each region of interest, the doctor can determine which regions of interest have image quality meeting the diagnosis requirements and which regions do not meet the diagnosis requirements, thereby making more informed diagnosis decisions.

In a possible implementation, the method further includes: simultaneously displaying the target medical image and the quality information.

In this implementation, the target medical image and corresponding quality information thereof may be displayed to the user simultaneously, so that the user can intuitively see the image content and related quality assessment data of the target medical image.

As an example of this implementation, on a user interface, the medical image may occupy a main display area, and the quality information may be presented in the form of a sidebar, a pop-up window, or an overlay.

As an example of this implementation, the quality information may include at least part of the following: a similarity between the current digital watermark and the original digital watermark, a resolution of the target medical image, a contrast of the target medical image, a compression ratio of the target medical image, and validity information of the target medical image. The quality information may be presented in the form of a numerical value, a graph, a color code, or other visual indications, so that the user can quickly grasp the image quality.

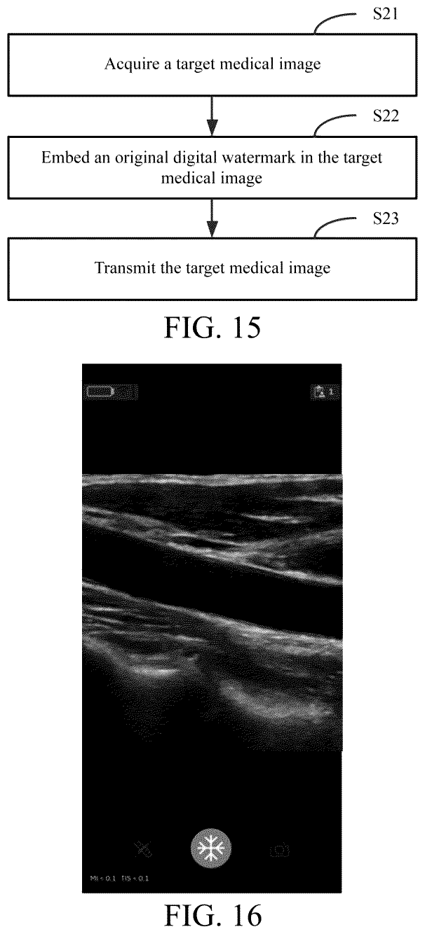

FIG. 15 illustrates another flowchart of the medical image processing method provided in an embodiment of the present disclosure. In a possible implementation, an executing entity of the medical image processing method may be a medical image processing apparatus. For example, the medical image processing method may be executed by a terminal device or other electronic devices. The terminal device may be a mobile phone, a tablet computer, a PC, a notebook computer, or the like. In a possible implementation, the executing entity of the medical image processing method may be a terminal device corresponding to a medical image examiner. For example, the medical image examiner may be a sonographer, a radiologist, etc. The terminal device corresponding to the observer may be any terminal device that can be operated by the observer. In some possible implementations, the medical image processing method may be implemented by a processor by invoking computer-readable instructions stored in a memory. As shown in FIG. 15, the medical image processing method includes step S21 to step S23.

In step S21, a target medical image is acquired.

In step S22, an original digital watermark is embedded in the target medical image.

In step S23, the target medical image is transmitted.

In a possible implementation, the acquiring a target medical image includes: performing imaging on a subject to be imaged to obtain imaging data; and processing the data to generate the target medical image.

The subject to be imaged may refer to a patient or an individual who needs to undergo an imaging examination. That is, the subject to be imaged may refer to a person who will undergo imaging of an internal structure of the body via a medical image device. In this implementation, the medical image device may be used to scan or image the subject to be imaged. During the imaging process, the medical image device may collect a series of imaging data. The imaging data, after undergoing certain processing, may be converted into a target medical image for doctor's diagnosis. The processing process may include parsing, reconstruction, enhancement, etc., of the data.

In the embodiments of the present disclosure, the image type of the target medical image may be an image or a video. In the case where the target medical image is a video, the original digital watermark may be embedded in the target medical image frame by frame, that is, the original digital watermark may be embedded in each frame of the target medical image.

Compared with a non-handheld medical image device, a medical image system based on a handheld medical image device can more easily disseminate medical information over the network. This may lead to unauthorized sharing, potential abuse of sensitive medical images, image tampering, and degradation of image quality, etc. In the embodiments of the present disclosure, it is beneficial for addressing these issues by embedding a digital watermark in the medical image. The digital watermark may be used for tracking, authentication, etc.

In a possible implementation, the embedding an original digital watermark in the target medical image includes: embedding an original digital watermark in the target medical image in response to a medical image save instruction, and saving the target medical image embedded with the original digital watermark.

In a possible implementation, the target medical image may be partitioned into a plurality of blocks by discrete wavelet transform (DWT). DWT is a mathematical transform, which can decompose an image into different frequency components for subsequent processing. After decomposition via DWT, medium-frequency and high-frequency regions may be selected as target regions for digital watermark embedding. The medium-frequency region contains most of the information of the image, and the high-frequency region contains edge and detail information. These regions are selected due to their insensitivity in the human visual system, thereby ensuring the invisibility of the digital watermark. In this implementation, DWT decomposition from level 2 to level 3 may be performed to obtain a more detailed image representation. Such multi-level decomposition helps to embed digital watermarks at different scales, thereby improving concealment and anti-attack capability of the digital watermark.

In a possible implementation, the embedding an original digital watermark in the target medical image in response to a medical image save instruction includes: in a security protection mode, in response to the medical image save instruction, embedding an original digital watermark in the target medical image. In this implementation, where the security protection mode is enabled, the digital watermark is automatically embedded when the medical image is saved.

In a possible implementation, the method further comprises: in response to an instruction to enable the security protection mode, enabling the security protection mode; or in response to an instruction to disable the security protection mode, disabling the security protection mode. In this implementation, a user can enable or disable the security protection mode at any time according to his/her own will.

In another possible implementation, the security protection mode may be enabled by default.

In a possible implementation, the embedding an original digital watermark in the target medical image includes: determining a region of interest of the target medical image, and embedding the original digital watermark in the region of interest in such a manner that the size of the original digital watermark matches the size of the region of interest.

In this implementation, before the original digital watermark is embedded in the target medical image, the region of interest may be determined in the target medical image. In medical image analysis, the entire image may contain a large amount of information, but not all of the information is necessary for diagnosis. The regions of interest are regions that are of particular concern to doctors or analysts in the medical image, and these regions may contain information that has a decisive influence on diagnosis, such as lesions, organs, tissues, or other key anatomical features.

As one example of this implementation, the regions of interest may be manually selected regions. When a medical image scan is performed, doctors can subjectively determine certain regions as regions of interest based on clinical experience and understanding of a specific disease condition. For example, during mammography, doctors may pay special attention to certain regions within breast tissues, as these regions may show abnormal densities or shapes. Doctors or technicians can manually mark regions of interest on the target medical image, so as to focus on these regions in subsequent analysis and diagnosis.

As another example of this implementation, the regions of interest may be automatically determined regions. In this example, computer-aided diagnosis (CAD) systems or deep learning algorithms may be utilized to automatically detect and identify specific features in the target medical image. These techniques may identify key anatomical structures, such as glands, blood vessels, bones, etc., and mark them as regions of interest.

In this implementation, after the region of interest in the target medical image is determined, the original digital watermark may be embedded into the region of interest in such a manner that the size of the original digital watermark matches the size of the region of interest. The size of the original digital watermark matching the size of the region of interest may indicate that the size of the original digital watermark is consistent with the size of the region of interest, that is, the original digital watermark is spatially completely adapted to the region of interest it is located in, without exceeding or falling short of the region of interest.

In this implementation, by embedding the original digital watermark only in the region of interest, it can be ensured that the most critical part in the target medical image is protected. This is crucial to prevent unauthorized modification or tampering of the target medical image, especially when important diagnostic decisions are involved. Additionally, by embedding the original digital watermark only in the region of interest, computing resources required for processing the entire target medical image can be reduced. This makes the entire process more efficient, especially in resource limited situations. Furthermore, since the original digital watermark is only embedded into the region of interest, the overall data size of the target medical image can be reduced, thereby optimizing the storage and transmission process. This is particularly important for medical applications that require remote transmission of images, such as remote diagnosis and medical counseling.

In a possible implementation, at least two regions of interest are included; and the original digital watermark is embedded in a merged region of the at least two regions of interest, alternatively, the original digital watermark is embedded in the at least two regions of interest, respectively.

As an example of this implementation, in the case where the target medical image includes at least two regions of interest, the original digital watermark may be embedded in a merged region of the at least two regions of interest.

In an example, for any two regions of interest in the target medical image, in the case where a distance between the two regions of interest is less than or equal to a preset distance threshold, the original digital watermark may be embedded in a merged region of the two regions of interest.

As another example of this implementation, in the case where the target medical image includes at least two regions of interest, the original digital watermark may be embedded in the at least two regions of interest, respectively. The original digital watermark being embedded in the at least two regions of interest, respectively, may represent that each of the at least two regions of interest includes one complete original digital watermark, that is, the original digital watermarks in different regions of interest are independent of each other.

In an example, for any two regions of interest in the target medical image, in the case where a distance between the two regions of interest is greater than a preset distance threshold, the original digital watermark may be embedded in the two regions of interest respectively.

In a possible implementation, the method further comprises: acquiring and displaying a medical image acquired by a medical image device; acquiring a screenshot image in response to a screenshot instruction; eliminating medical-related information in the screenshot image to obtain a screenshot-protected image; and storing the screenshot-protected image.

A screenshot function of a mobile terminal increases the possibility that the medical image is tampered with. In this implementation, the screenshot-protected image does not include the medical-related information, thereby resolving a problem that security of patient information is threatened due to the screenshot function of the mobile terminal.

In a possible implementation, the eliminating medical-related information in the screenshot image to obtain a screenshot-protected image includes: in response to being in a security protection mode, eliminating the medical-related information from the screenshot image to obtain the screenshot-protected image.

In this implementation, when the security protection mode is enabled, screenshots of the medical image will be prohibited.

In a possible implementation, the eliminating medical-related information in the screenshot image to obtain a screenshot-protected image includes: replacing the screenshot image with a preset screenshot-protected image, where the preset screenshot-protected image does not contain the medical-related information.

As an example of this implementation, the preset screenshot-protected image may be a completely black image.

FIG. 16 illustrates a schematic diagram of a medical image displayed on a terminal device corresponding to a medical image examiner in the medical image processing method provided in an embodiment of the present disclosure.

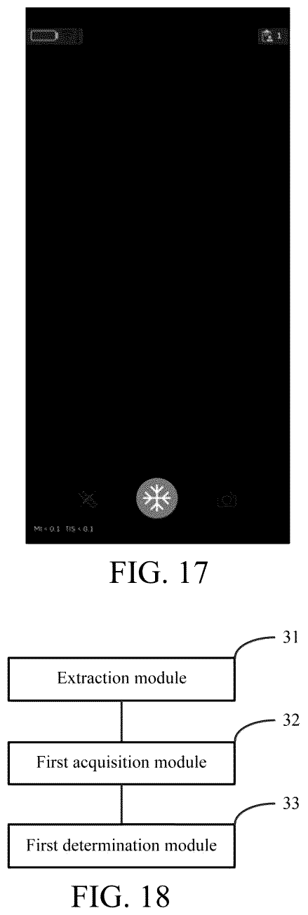

FIG. 17 illustrates a schematic diagram of a screenshot-protected image in the medical image processing method provided in an embodiment of the present disclosure. In the example shown in FIG. 17, the screenshot-protected image is a completely black image, and does not include any medical-related information.

It can be understood that all the foregoing method embodiments mentioned in the present disclosure may be combined with each other to form a combined embodiment without departing from the principle or logic, which will not be described in detail in the present disclosure due to limited space. It can be understood by those skilled in the art that, in the above methods described in the detailed embodiments, the specific order in which each step is performed should be determined by the function and possible inherent logic thereof.

In addition, the present disclosure also provides a medical image processing apparatus, an electronic device, a computer-readable storage medium, and a computer program product, all of which can be used to implement any one of the medical image processing methods provided by the present disclosure. For corresponding technical solutions and technical effects, reference may be made to corresponding descriptions in the method sections, and details will not be described herein again.

FIG. 18 illustrates a block diagram of a medical image processing apparatus provided in an embodiment of the present disclosure. As shown in FIG. 18, the medical image processing apparatus includes:
an extraction module 31, used to extract a current digital watermark in a target medical image;
a first acquisition module 32, used to acquire an original digital watermark corresponding to the target medical image; and
a first determination module 33, used to compare the current digital watermark with the original digital watermark to determine quality information of the target medical image.

In a possible implementation, the extraction module 31 is used to:
in response to an instruction to open the target medical image, automatically extract the current digital watermark in the target medical image.

In a possible implementation, the first determination module 33 is used to:
determine a similarity between the current digital watermark and the original digital watermark; and
determine the quality information of the target medical image based on the similarity.

In a possible implementation, the quality information includes validity information of the target medical image;
the first determination module 33 is used to:
in response to the similarity being greater than or equal to a first preset similarity threshold, determine that the validity information of the target medical image is valid; or,
in response to the similarity being less than the first preset similarity threshold, determine that the validity information of the target medical image is invalid.

In a possible implementation, the apparatus further includes:
a second determination module, used to determine whether an image quality value of the target medical image is less than a quality threshold; and
when the image quality value is less than the quality threshold, the quality information includes a low quality cause of the target medical image.

In a possible implementation, when the image quality value is less than the quality threshold, the first determination module 33 is used to:
in response to the similarity being greater than or equal to a second preset similarity threshold, determine that the low quality cause of the target medical image includes an imaging cause;
or,
in response to the similarity being less than the second preset similarity threshold, determine that the low quality cause of the target medical image comprises a transmission cause and/or a compression cause.

In a possible implementation, the current digital watermark is embedded in a region of interest of the target medical image, and the size of the current digital watermark matches the size of the region of interest.

In a possible implementation, at least two regions of interest are included; and
the current digital watermark is embedded in a merged region of the at least two regions of interest,
or,
the current digital watermark is embedded in the at least two regions of interest, respectively.

In a possible implementation, the quality information comprises:
region quality information in one-to-one correspondence with the at least two regions of interest.

In a possible implementation, the shape of the original digital watermark corresponds to the shape of the target medical image.

In a possible implementation, the apparatus further includes:
a display module, used to simultaneously display the target medical image and the quality information.

In a possible implementation, the target medical image is obtained through transmission.

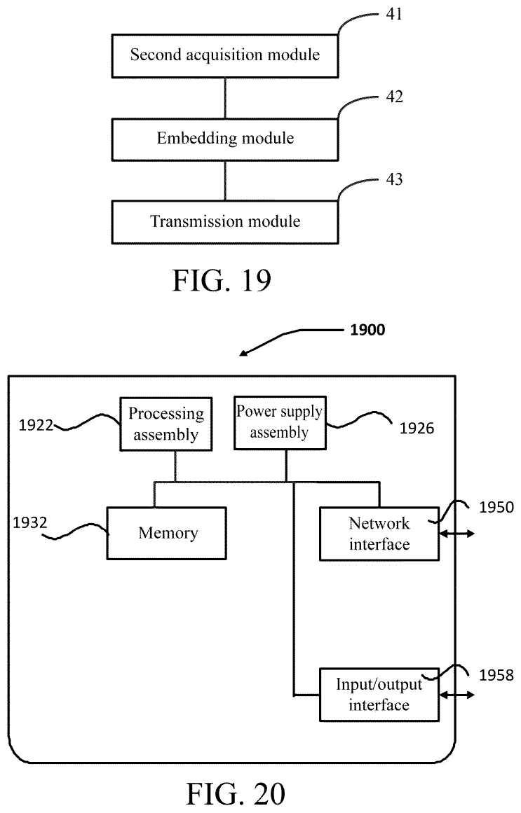

FIG. 19 illustrates another block diagram of the medical image processing apparatus provided in an embodiment of the present disclosure. As shown in FIG. 19, the medical image processing apparatus includes:
a second acquisition module 41, used to acquire a target medical image;
an embedding module 42, used to embed an original digital watermark in the target medical image; and
a transmission module 43, used to transmit the target medical image.

In a possible implementation, the second acquisition module 41 is used to:
perform imaging on a subject to be imaged to obtain imaging data; and
process the data to generate the target medical image.

In a possible implementation, the embedding module 42 is used to:
determine a region of interest of the target medical image, and embed the original digital watermark in the region of interest in such a manner that the size of the original digital watermark matches the size of the region of interest.

In some embodiments, the function of the apparatus provided in the embodiments of the present disclosure or the modules included in the apparatus may be used to perform the method described in the foregoing method embodiments, and for specific implementation and technical effects thereof, reference may be made to the description of the foregoing method embodiments, which are not described herein again for brevity.

An embodiment of the present disclosure further provides a computer-readable storage medium, having computer program instructions stored thereon, wherein the computer program instructions, when executed by a processor, implement the above method. The computer-readable storage medium may be a non-volatile computer-readable storage medium or a volatile computer-readable storage medium.

An embodiment of the present disclosure further provides a computer program, including computer-readable code, wherein when the computer-readable code is run in an electronic device, a processor in the electronic device performs the above method.

An embodiment of the present disclosure further provides a computer program product, including computer-readable code or a non-volatile computer-readable storage medium carrying computer-readable code, wherein when the computer-readable code is run in an electronic device, a processor in the electronic device performs the above method.

An embodiment of the present disclosure further provides an electronic device, including: one or more processors; and a memory for storing executable instructions, wherein the one or more processors are configured to invoke the executable instructions stored by the memory to perform the above method.

The electronic device may be provided as a terminal, a server, or a device in another form.

FIG. 20 illustrates a block diagram of an electronic device 1900 provided in an embodiment of the present disclosure. For example, the electronic device 1900 may be provided as a terminal or a server. Referring to FIG. 20, the electronic device 1900 includes a processing assembly 1922 that further includes one or more processors, and a memory resource represented by a memory 1932 for storing instructions that can be executed by the processing assembly 1922, such as an application. The application stored in the memory 1932 can include one or more modules each corresponding to a set of instructions. In addition, the processing assembly 1922 is configured to execute instructions to perform the above method.

The electronic device 1900 can further include a power supply assembly 1926 configured to perform power management of the electronic device 1900, a wired or wireless network interface 1950 configured to connect the electronic device 1900 to a network, and an input/output (I/O) interface 1958. The electronic device 1900 can operate on the basis of an operating system stored in the memory 1932, for example, a Microsoft server operating system (Windows Server™), a graphical user interface-based operating system (Mac OS X™) provided by Apple Inc., a multi-user multi-process computer operating system (Unix™), a free and open source Unix-like operating system (Linux™), an open source Unix-like operating system (FreeBSD™) or the like.

In an exemplary embodiment, a non-volatile computer-readable storage medium is also provided, such as the memory 1932 including computer program instructions that are executable by the processing assembly 1922 of the electronic device 1900 to perform the above method.

The present disclosure may be a system, a method and/or a computer program product. The computer program product may include a computer-readable storage medium, having computer-readable program instructions thereon for causing a processor to implement various aspects of the present disclosure.

The computer-readable storage medium can be a tangible device that can hold and store instructions used by an instruction execution device. The computer-readable storage medium may be, for example, but is not limited to an electrical storage device, a magnetic storage device, an optical storage device, an electromagnetic storage device, a semiconductor memory device, or any suitable combination thereof. More specific examples (a non-exhaustive list) of the computer-readable storage medium include: a portable computer disk, a hard disk, a random access memory (RAM), a read only memory (ROM), a erasable programmable read only memory (EPROM or flash memory), a static random access memory (SRAM), a portable compact disc read-only memory (CD-ROM), a digital versatile disk (DVD), a memory stick, a floppy disk, mechanical coding equipment, such as a punch card with instructions stored thereon or a structure of bumps within recessions, and any suitable combination thereof. The computer-readable storage medium used herein is not interpreted as transient signals themselves, such as radio waves or other freely propagated electromagnetic waves, electromagnetic waves propagated through a waveguide or other transmission media (e.g., light pulses passing through a fiber optic cable), or electrical signals transmitted through electric wires.

The computer-readable program instructions described herein may be downloaded from a computer-readable storage medium to various computing/processing devices or downloaded to an external computer or external storage device via a network such as the Internet, a local area network, a wide area network and/or a wireless network. The network may include copper transmission cables, fiber transmission, wireless transmission, routers, firewalls, switches, gateway computers, and/or edge servers. A network adapter card or a network interface in each computing/processing device receives computer-readable program instructions from the network and forwards the computer-readable program instructions, for storing them in a computer-readable storage medium in each computing/processing device.

Computer program instructions for performing the operations of the present disclosure can be assembly instructions, instruction set architecture (ISA) instructions, machine instructions, machine related instructions, microcode, firmware instructions, state setting data, or source code or object code written in any combination of one or more programming languages, the programming language including object oriented programming languages such as Smalltalk, C++ and the like, and conventional procedural programming languages such as the “C” language or similar programming languages. The computer-readable program instructions can be executed entirely or partly on a user computer, executed as a stand-alone software package, executed partly on a user computer and partly on a remote computer, or executed entirely on a remote computer or server. In the case of a remote computer, the remote computer may be connected to the user's computer through any kind of network, including a local area network (LAN) or a wide area network (WAN). Alternatively, it can be connected to an external computer (for example, using an Internet service provider to connect via the Internet). In some embodiments, an electronic circuit, for example, a programmable logic circuit, a field-programmable gate array (FPGA), or a programmable logic array (PLA), may execute the computer-readable program instructions by utilizing state information of the computer-readable program instructions to personalize the electronic circuit, in order to implement various aspects of the present disclosure

The aspects of the present disclosure are described herein with reference to the flowcharts and/or block diagrams of the methods, apparatuses (systems), and computer program products according to the embodiments of the present disclosure. It should be understood that each block of the flowcharts and/or block diagrams and combinations of various blocks in the flowcharts and/or block diagrams can be implemented by computer-readable program instructions.

These computer-readable program instructions may be provided to a processor of a general-purpose computer, a special-purpose computer, or other programmable data processing apparatuses, to produce a machine, so that these instructions, when executed by the processor of the computer or other programmable data processing apparatuses, produce an apparatus for implementing the functions/actions specified in one or more blocks of the flowcharts and/or block diagrams. Also, these computer-readable program instructions may be stored in a computer-readable storage medium. These instructions cause a computer, a programmable data processing device, and/or other devices to work in a specific manner. Thus, the computer-readable medium storing the instructions includes an artifact, including instructions that implement various aspects of the functions/actions specified in one or more the flowcharts and/or block diagrams.

The computer-readable program instructions may also be loaded onto a computer, other programmable data processing apparatuses, or other devices, such that the computer, other programmable data processing apparatuses or other devices perform a series of operational steps, to generate a computer-implemented process, such that the functions/actions specified in one or more of the flowcharts and/or block diagrams are implemented by the instructions executed on the computer, other programmable data processing apparatuses, or other devices.

The flowcharts and block diagrams in the accompanying drawings illustrate system architectures, functions, and operations of possible implementations of the system, method, and computer program product according to a plurality of embodiments of the present disclosure. In this regard, each block in the flowcharts or block diagrams may represent a portion of a module, program segment, or instruction that contains one or more executable instructions for implementing the specified logical functions. In some alternative implementations, the functions denoted in the blocks can also occur in a different order than that illustrated in the drawings. For example, two consecutive blocks can actually be performed substantially in parallel, and sometimes can also be performed in a reverse order, depending upon the functions involved. It should also be noted that each block of the block diagrams and/or flowcharts, and combinations of blocks in the block diagrams and/or flowcharts can be implemented in a dedicated hardware-based system that performs the specified function or action, or can be implemented by a combination of dedicated hardware and computer instructions.

The computer program product may be embodied in hardware, software, or a combination thereof. In an alternative embodiment, the computer program product is embodied as a computer storage medium, and in another alternative embodiment, the computer program product is embodied as a software product, such as a software development kit (SDK) or the like.

The above description of the various embodiments tends to emphasize differences between the various embodiments, and reference may be made therebetween for the same features or similarities thereof, which will not be described in detail herein for the sake of brevity.

If the technical solution of the embodiments of the present disclosure involves personal information, a product to which the technical solution of the embodiments of the present disclosure is applied has explicitly performed notification of a personal information processing rule before processing the personal information, and has acquired individual self-consent. If the technical solution of the embodiments of the present disclosure involves sensitive personal information, a product to which the technical solution of the embodiments of the present disclosure is applied has acquired individual consent before processing the sensitive personal information, and also satisfies the requirement of “explicit consent”. For example, at a personal information collection apparatus such as a camera, a clear and obvious sign is provided to state that a personal information collection range has been entered, and personal information will be collected. If a person voluntarily enters the collection range, it is regarded as consent to collect personal information thereof. Alternatively, if notification of a personal information processing rule is performed on a personal information processing apparatus by using an obvious sign/information, personal authorization is acquired by means of pop-up information or by inviting the individual to upload personal information thereof. The personal information processing rule may include information such as a personal information handler, a personal information processing purpose, a processing method, and a type of personal information to be processed.

The embodiments of the present disclosure have been described above. The foregoing description is illustrative rather than limiting, and is not limited to the disclosed embodiments. Many modifications and variations are apparent to those of ordinary skill in the art without departing from the scope and spirit of the embodiments illustrated. The selection of terms used herein is intended to best explain the principles, practical applications, or improvements to the techniques in the market of the embodiments, or to enable others of ordinary skill in the art to understand the embodiments disclosed herein.

1. A medical image processing method, comprising:
extracting a current digital watermark in a target medical image;
acquiring an original digital watermark corresponding to the target medical image; and
comparing the current digital watermark with the original digital watermark; and
determining quality information of the target medical image based on the comparison.

2. The method according to claim 1, wherein the extracting a current digital watermark in a target medical image comprises:
in response to an instruction to open the target medical image, automatically extracting the current digital watermark in the target medical image.

3. The method according to claim 1, wherein the determining quality information of the target medical image comprises:
determining a similarity between the current digital watermark and the original digital watermark; and
determining the quality information of the target medical image based on the similarity.

4. The method according to claim 3, wherein the quality information comprises validity information of the target medical image; and
the determining the quality information of the target medical image based on the similarity comprises:
in response to the similarity being greater than or equal to a first preset similarity threshold, determining that the validity information of the target medical image is valid; or,
in response to the similarity being less than the first preset similarity threshold, determining that the validity information of the target medical image is invalid.

5. The method according to claim 3, further comprising:
determining whether an image quality value of the target medical image is less than a quality threshold; and
when the image quality value is less than the quality threshold, the quality information comprising a low quality cause of the target medical image.

6. The method according to claim 5, wherein when the image quality value is less than the quality threshold, the determining quality information of the target medical image comprises:
in response to the similarity being greater than or equal to a second preset similarity threshold, determining that the low quality cause of the target medical image comprises an imaging cause;
or,
in response to the similarity being less than the second preset similarity threshold, determining that the low quality cause of the target medical image comprises a transmission cause and/or a compression cause.

7. The method according to claim 1, wherein the current digital watermark is embedded in a region of interest of the target medical image, and the size of the current digital watermark matches the size of the region of interest.

8. The method according to claim 7, wherein
at least two regions of interest are comprised; and
the current digital watermark is embedded in a merged region of the at least two regions of interest,
or,
the current digital watermark is embedded in the at least two regions of interest, respectively.

9. The method according to claim 8, wherein the quality information comprises:
region quality information in one-to-one correspondence with the at least two regions of interest.

10. The method according to claim 1, wherein the shape of the original digital watermark corresponds to the shape of the target medical image.

11. The method according to claim 1, further comprising:
simultaneously displaying the target medical image and the quality information.

12. The method according to claim 1, wherein:
the target medical image is obtained through transmission.

13. A non-transitory computer readable medium storing thereon instruction that, when executed by a processor, cause the processor to:
extract a current digital watermark in a target medical image;
acquire an original digital watermark corresponding to the target medical image; and
compare the current digital watermark with the original digital watermark; and
determine quality information of the target medical image based on the comparison.

14. A medical image processing apparatus, comprising:
a memory storing instructions; and
a processor configured to execute the instructions to:
extract a current digital watermark in a target medical image;
acquire an original digital watermark corresponding to the target medical image; and
compare the current digital watermark with the original digital watermark to determine quality information of the target medical image.

15. The medical image processing apparatus of claim 14, wherein the processor is further configured to execute the instructions to:
acquire a target medical image;
embed an original digital watermark in the target medical image; and
transmit the target medical image.
